(19) United States
(12) Patent Application Publication
(10) Pub. No.: US 2008/0319245 A1
(43) Pub. Date: Dec. 25, 2008

(54) Catalyst and Process for Producing Liquefied Petroleum Gas

(76) Inventors: Kaoru Fujimoto, Kitakyushu-shi (JP); Kenji Asami, Kitakyushu-shi (JP); Xiaohong Li, Kitakyushu-shi (JP); Sachio Asaoka, Kitakyushu-shi (JP); Qianwen Zhang, Kitakyushu-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 11/573,242

(22) PCT Filed: May 30, 2005
(86) PCT No.: PCT/JP2005/009874

§ 371 Date: Feb. 5, 2007

(30) Foreign Application Priority Data
Aug. 10, 2004 (JP) 2004-233327

(51) Int. Cl. C07C 1/00; B01J 29/06; B01J 29/072

(52) U.S. Cl. 585/733; 502/60; 502/74

(57) ABSTRACT

A catalyst for producing a liquefied petroleum gas according to the present invention comprises a methanol synthesis catalyst component in which an olefin-hydrogenation catalyst component is supported on a Zn—Cr-based methanol synthesis catalyst, and a zeolite catalyst component. It can be used in a reaction of carbon monoxide and hydrogen to give a hydrocarbon containing propane or butane as a main component, i.e., a liquefied petroleum gas, with high activity, high selectivity and high yield. Furthermore, the catalyst has a longer catalyst life with less deterioration.

### TECHNICAL FIELD

This invention relates to a catalyst for producing a liquefied petroleum gas containing propane or butane as a main component by reacting carbon monoxide with hydrogen.

This invention also relates to a process for producing a liquefied petroleum gas containing propane or butane as a main component from a synthesis gas using the catalyst. This invention also relates to a process for producing a liquefied petroleum gas containing propane or butane as a main component from a carbon-containing starting material such as a natural gas using the catalyst.

### BACKGROUND ART

Liquefied petroleum gas (LPG) is a liquefied petroleum-based or natural-gas-based hydrocarbon which is gaseous at an ambient temperature under an atmospheric pressure by compression while optionally cooling, and the main component of it is propane or butane. LPG is advantageously transportable because it can be stored or transported in a liquid form. Thus, in contrast with a natural gas that requires a pipeline for supply, it has a characteristic that it can be filled in a container to be supplied to any place. For that reason, LPG comprising propane as a main component, i.e., propane gas has been widely used as a fuel for household and business use. At present, propane gas is supplied to about 25 million households (more than 50% of the total households) in Japan. In addition to household and business use, LPG is used as a fuel for a portable product such as a portable gas burner and a disposable lighter (mainly, butane gas), an industrial fuel and an automobile fuel.

Conventionally, LPG has been produced by 1) collection from a wet natural gas, 2) collection from a stabilization (vapor-pressure regulating) process of crude petroleum, 3) separation and extraction of a product in, for example, a petroleum refining process, or the like.

LPG, in particular propane gas used as a household/business fuel, can be expected to be in great demand in the future. Thus, it may be very useful to establish an industrially practicable and new process for producing LPG.

As a process for producing LPG, Patent document 1 discloses that a synthesis gas consisting of hydrogen and carbon monoxide is reacted in the presence of a mixed catalyst obtained by physically mixing a methanol synthesis catalyst such as a Cu—Zn-based catalyst, a Cr—Zn-based catalyst and a Pd-based catalyst, specifically a CuO—ZnO—Al₂O₃ catalyst, a Pd/SiO₂ catalyst or a Cr—Zn-based catalyst with a methanol conversion catalyst composed of a zeolite having an average pore size of about 10 Å (1 nm) or more, specifically a Y-type zeolite, to give a liquefied petroleum gas or a mixture of hydrocarbons similar in composition to LPG.

The catalyst described in the above-mentioned Patent document 1, however, does not necessarily show sufficient performance.

For example, a catalyst consisting of Pd/SiO₂ and a Y-type zeolite is less active and gives a lower yield of a hydrocarbon, in which a ratio of propane (C3) and butane (C4) is lower. A catalyst consisting of Pd/SiO₂ and a dealuminated Y-type zeolite with SiO₂/Al₂O₃=7.6 treated with steam at 450° C. for 2 hours has a higher activity and gives a higher yield of a hydrocarbon, in which a ratio of propane (C3) and butane (C4) is higher, but it may not have sufficient performance particularly in terms of activity and yield of a hydrocarbon.

Furthermore, although the above-mentioned Patent document 1 does not disclose the amount of supported Pd in the Pd-based methanol synthesis catalyst, i.e., Pd/SiO₂ catalyst, it is generally about 4% by weight, and high-priced Pd is used in relatively large quantities. Therefore, a catalyst consisting of a Pd-based methanol synthesis catalyst (Pd/SiO₂) and a Y-type zeolite may be unfavorable in terms of catalyst cost.

On the other hand, as a general trend, a catalyst consisting of a Cu—Zn-based catalyst (a copper-zinc-alumina mixed oxide and a commercially available catalyst for methanol synthesis at a low pressure) and a Y-type zeolite has a higher activity and gives a higher yield of a hydrocarbon, in which a ratio of propane (C3) and butane (C4) is higher, in comparison with a catalyst consisting of Pd/SiO₂ and a Y-type zeolite. Among them, a catalyst consisting of a Cu—Zn-based catalyst and a dealuminated Y-type zeolite with SiO₂/Al₂O₃=7.6 treated with steam at 450° C. for 2 hours has a high activity and gives a high yield of a hydrocarbon, in which a ratio of propane (C3) and butane (C4) is high. However, a catalyst consisting of a Cu—Zn-based catalyst and a Y-type zeolite may be significantly deteriorated with time, and thus, it may not have a sufficiently long catalyst life in general. It is, therefore, difficult to stably produce LPG in a high yield for a long period using the catalyst.

The catalyst consisting of the Zn—Cr-based catalyst and the Y-type zeolite described in the above-mentioned Patent document 1 has a lower activity, lower yield of a hydrocarbon, and lower selectivity of propane and butane, in comparison with a catalyst consisting of Pd/SiO₂ and a Y-type zeolite. It is mentioned in Patent document 1 that a Zn—Cr-based catalyst may not sufficiently function as a methanol synthesis catalyst under the LPG synthesis reaction condition.

Thus, it has been needed further improvement of a catalyst for producing a liquefied petroleum gas in order to practically use a process for producing LPG from a synthesis gas and a process for producing LPG from a carbon-containing starting material such as a natural gas.

As a process for producing LPG, Non-patent document 1 discloses that a hybrid catalyst consisting of a methanol synthesis catalyst such as a 4 wt % Pd/SiO₂, a Cu—Zn—Al mixed oxide {Cu:Zn:Al=40:23:37 (atomic ratio)} or a Cu-based low-pressure methanol synthesis catalyst (Trade name: BASF S3-85) and a high-silica Y-type zeolite with SiO₂/Al₂O₃=7.6 treated with steam at 450° C. for 1 hour can be used to produce C2 to C4 paraffins in a selectivity of 69 to 85% via methanol and dimethyl ether from a synthesis gas. However, as is for the catalyst described in the above-mentioned Patent document 1, the catalyst described in Non-patent document 1 may not show sufficient performance.

Furthermore, in the catalyst described in the above-mentioned Non-patent document 1, the amount of supported Pd in the Pd-based methanol synthesis catalyst, i.e., Pd/SiO₂ catalyst is 4% by weight, and high-priced Pd is used in relatively large quantities. Therefore, the catalyst consisting of 4 wt % Pd/SiO₂ and Y-type zeolite described in Non-patent document 1 may be unfavorable in terms of catalyst cost.

In addition, Non-patent document 2 discloses that a hybrid catalyst consisting of Pd—SiO₂ or Pd, Ca—SiO₂, and a P-zeolite or a USY-type zeolite can be used to produce LPG from a synthesis gas. In the catalyst described in Non-patent document 2, however, the amount of supported Pd in Pd—SiO₂ or Pd, Ca—SiO₂, which is a methanol synthesis catalyst, is 4% by weight, and high-priced Pd is used in relatively large quantities. Therefore, the catalyst consisting of Pd—SiO₂ or Pd, Ca—SiO₂ and a zeolite described in Non-patent document 2 may be also unfavorable in terms of catalyst cost.

### LIST OF REFERENCES

Patent document 1: Japanese Patent Laid-open Publication No. 61-23688;

Non-patent document 1: “Selective Synthesis of LPG from Synthesis Gas”, Kaoru Fujimoto et al., Bull. Chem. Soc. Jpn., 58, p. 3059-3060 (1985);

Non-patent document 2: “Synthesis of LPG from Synthesis Gas with Hybrid Catalyst”, Qianwen Zhang et al., Dai 33 Kai Sekiyu Sekiyu Kagaku Toronkai Koen Yoshi (the summaries of the 33th Petroleum and Petrochemistry Discussion), p. 179-180, Nov. 17, 2003.

### DISCLOSURE OF THE INVENTION

### Problems to be Solved by the Invention

An objective of this invention is to provide a less deteriorative catalyst for producing a liquefied petroleum gas with a longer catalyst life, which enables the production of a hydrocarbon containing propane or butane as a main component, i.e., a liquefied petroleum gas (LPG), by reacting carbon monoxide and hydrogen, with high activity, high selectivity and high yield.

Another objective of this invention is to provide a process for stably producing LPG with a high concentration of propane and/or butane from a synthesis gas in a high yield for a long period, using the catalyst. A further objective of this invention is to provide a process for stably producing LPG with a high concentration of propane and/or butane from a carbon-containing starting material such as a natural gas in a high yield for a long period.

### Means for Solving the Problems

The present invention provides a catalyst for producing a liquefied petroleum gas, which is used for producing a liquefied petroleum gas containing propane or butane as a main component by reacting carbon monoxide and hydrogen, comprising

a methanol synthesis catalyst component in which an olefin-hydrogenation catalyst component is supported on a Zn—Cr-based methanol synthesis catalyst; and

a zeolite catalyst component.

Herein, an “olefin-hydrogenation catalyst component” means a compound which can act as a catalyst in a hydrogenation reaction of an ole fin into a paraffin. A “Zn—Cr-based methanol synthesis catalyst” means a compound containing Zn and Cr which can act as a catalyst in the reaction of CO+2H₂−CH₃OH. And a “zeolite catalyst component” means a zeolite which can act as a catalyst in a condensation reaction of methanol into a hydrocarbon and/or a condensation reaction of dimethyl ether into a hydrocarbon.

Moreover, the present invention provides a process for producing a liquefied petroleum gas, comprising a step of:

reacting carbon monoxide and hydrogen in the presence of the catalyst for producing a liquefied petroleum gas as described above, whereby producing a liquefied petroleum gas containing propane or butane as a main component.

The present invention also provides a process for producing a liquefied petroleum gas, comprising a step of:

feeding a synthesis gas to a catalyst layer comprising the catalyst for producing a liquefied petroleum gas as described above, whereby producing a liquefied petroleum gas containing propane or butane as a main component (Liquefied petroleum gas production process).

Moreover, the present invention provides a process for producing a liquefied petroleum gas, comprising:

(1) a step of producing a synthesis gas from a carbon-containing starting material and at least one selected from the group consisting of H₂O, O₂ and CO₂ (Synthesis gas production process); and

(2) a step of producing a liquefied petroleum gas wherein the synthesis gas is fed to a catalyst layer comprising the catalyst for producing a liquefied petroleum gas as described above, whereby producing a liquefied petroleum gas containing propane or butane as a main component (Liquefied petroleum gas production process).

Herein, “synthesis gas” means a mixed gas comprising hydrogen and carbon monoxide, and is not limited to a mixed gas consisting of hydrogen and carbon monoxide. A synthesis gas may be, for example, a mixed gas comprising carbon dioxide, water, methane, ethane, ethylene and so on. A synthesis gas produced by reforming a natural gas generally contains, in addition to hydrogen and carbon monoxide, carbon dioxide and water vapor. A synthesis gas may be a coal gas produced by coal gasification, or a water gas produced from a coal coke.

### EFFECT OF THE INVENTION

A catalyst for producing a liquefied petroleum gas according to this invention comprises a methanol synthesis catalyst component in which an olefin-hydrogenation catalyst component is supported on a Zn—Cr-based methanol synthesis catalyst, and a zeolite catalyst component.

A preferable methanol synthesis catalyst component is a catalyst in which 0.005 to 5 wt %, more preferably 0.5 to 5 wt % of the olefin-hydrogenation catalyst component is supported on a Zn—Cr-based methanol synthesis catalyst. Among others, a particularly preferable methanol synthesis catalyst component is a catalyst in which 0.005 to 5 wt %, more preferably 0.5 to 5 wt % of Pd is supported on a composite oxide containing Zn and Cr. A preferable zeolite catalyst component is a β-zeolite, particularly preferably a proton-type β-zeolite with a SiO₂/Al₂O₃ molar ratio of 10 to 150. Another particularly preferable zeolite catalyst component is a Pd-supported β-zeolite with a SiO₂/Al₂O₃ molar ratio of 10 to 150 in which the amount of supported Pd is 3% by weight or less.

The catalyst for producing a liquefied petroleum gas according to this invention enables the production of a hydrocarbon containing propane or butane as a main component, i.e., a liquefied petroleum gas (LPG) with high activity, high selectivity and a high yield, by reacting carbon monoxide and hydrogen, and has a longer catalyst life with less deterioration.

By reacting carbon monoxide and hydrogen in the presence of the catalyst according to this invention, the reaction represented by the following formula (I) may proceed to give LPG containing propane or butane as a main component.

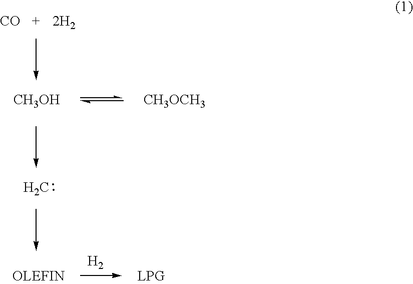

First, on the methanol synthesis catalyst component, methanol is formed from carbon monoxide and hydrogen, while dimethyl ether is also formed by dehydro-dimerization of methanol. Then, methanol thus formed is converted to a lower-olefin hydrocarbon comprising propylene or butene as a main component at an active site in a pore in the zeolite catalyst component. In the reaction, methanol would be dehydrated to give a carbene (H₂C:), which is subjected to polymerization to give a lower olefin. The lower olefin thus generated is released from the pore in the zeolite catalyst component and is rapidly hydrogenated on the methanol synthesis catalyst component to give a paraffin comprising propane or butane as a main component, i.e., LPG.

Herein, a “methanol synthesis catalyst component” means a compound which can act as a catalyst in the reaction of CO+2H₂→CH₃OH. And a “zeolite catalyst component” means a zeolite which can act as a catalyst in a condensation reaction of methanol into a hydrocarbon and/or a condensation reaction of dimethyl ether into a hydrocarbon.

As a methanol synthesis catalyst, a Cu—Zn-based catalyst (a composite oxide containing Cu and Zn) and a Zn—Cr-based catalyst (a composite oxide containing Zn and Cr) are widely used. However, sufficient catalyst performance cannot be always achieved when using a Cu—Zn-based methanol synthesis catalyst or a conventional Zn—Cr-based methanol synthesis catalyst as a methanol synthesis catalyst component in a catalyst for producing a liquefied petroleum gas, which is used for producing LPG by reacting carbon monoxide and hydrogen.

In addition, a Pd-based catalyst is also known to act as a catalyst in the methanol synthesis reaction (CO+2H₂→CH₃OH).

The reaction of carbon monoxide and hydrogen for producing LPG depends on a variety of factors. Therefore, the reason why the catalyst for producing a liquefied petroleum gas of this invention exhibits excellent performance is not clear, but the followings might be assumed.

A reaction of carbon monoxide and hydrogen for forming methanol

(CO+2H₂→CH₃OH) is an equilibrium reaction. And, the equation: CO+2H₂═CH₃OH+100 kJ indicates that the equilibrium of methanol formation is more advantageous as a temperature is lower. However, when reacting carbon monoxide and hydrogen to produce LPG, methanol formed on a methanol synthesis catalyst component is rapidly converted to a lower-olefin hydrocarbon at an active site in a pore in the zeolite catalyst component. There are, therefore, substantially no restrictions to the equilibrium of methanol formation. Thus, it is not necessarily required to conduct the reaction at a lower temperature for achieving a sufficiently high yield. On the other hand, it is advantageous to conduct the reaction at a higher temperature in terms of a reaction rate. In the light of activity of the zeolite catalyst component, it is desirable that the methanol synthesis catalyst component has great heat-resistance to some degree. Specifically, the methanol synthesis catalyst component can be used preferably at 270° C. or higher, more preferably 300° C. or higher, further preferably 320° C. or higher.

Among methanol synthesis catalysts, a Cu—Zn-based catalyst is generally used at a relatively lower temperature (about 230 to 300° C.) and its heat resistance is not so higher than other methanol synthesis catalysts. When producing LPG by reacting carbon monoxide and hydrogen at an elevated temperature for achieving higher activity and higher yield, it is not necessarily preferable to use a conventional Cu—Zn-based catalyst as a methanol synthesis catalyst component.

On the other hand, among methanol synthesis catalysts, a Zn—Cr-based catalyst is generally used at a relatively higher temperature (about 250 to 400° C.). There appears to be no particular problem in using a Zn—Cr-based catalyst as a methanol synthesis catalyst component when making a reaction temperature higher.

However, when producing LPG by reacting carbon monoxide and hydrogen, a methanol synthesis catalyst component is required to act as a catalyst in a hydrogenation reaction of an olefin into a paraffin. A conventional Zn—Cr-based catalyst, however, does not have sufficient hydrogenating ability. Thus, when producing LPG by reacting carbon monoxide and hydrogen, it may not be preferable to use a conventional Zn—Cr-based catalyst as a methanol synthesis catalyst component.

According to this invention, a methanol synthesis catalyst component to be used is a catalyst in which an olefin-hydrogenation catalyst component as a promoter is added to a conventional Zn—Cr-based methanol synthesis catalyst, which does not have sufficient hydrogenating ability, thereby giving the hydrogenating ability required for LPG synthesis, resulting in both high thermal stability and adequate hydrogenating ability of the methanol synthesis catalyst component. Owing to its high thermal stability and hydrogenating ability, a catalyst in which an olefin-hydrogenation catalyst component is supported on a Zn—Cr-based methanol synthesis catalyst may be suitable as a methanol synthesis catalyst component in a catalyst used in producing a liquefied petroleum gas by reacting carbon monoxide and hydrogen, particularly at an elevated reaction temperature.

In this invention, it is essential that an olefin-hydrogenation catalyst component be supported on a Zn—Cr-based methanol synthesis catalyst. The remarkable effect of the present invention may not be obtained when using a catalyst comprising a Zn—Cr-based methanol synthesis catalyst and a Pd-supported β-zeolite, although Pd is an olefin-hydrogenation catalyst component.

A Pd-based methanol synthesis catalyst has high thermal stability and hydrogenating ability, and it may be suitable as a methanol synthesis catalyst component, which is used in combination with β-zeolite, particularly when the reaction temperature is higher. As described above, however, the Pd-based methanol synthesis catalyst contains a relatively large amount of high-priced Pd, and therefore, a catalyst for producing a liquefied petroleum gas which comprises a Pd-based methanol synthesis catalyst as a methanol synthesis catalyst component may be disadvantageous cost-wise, in comparison with the catalyst of this invention.

On the other hand, the example of the zeolite catalyst component used in combination with the methanol synthesis catalyst of this invention may include various zeolites such as Y-type, ZSM-5, mordenite, SAPO-34 and MCM-22 zeolites. However, not all of the zeolites can give a catalyst with excellent performance.

A preferable zeolite catalyst component may be a middle-pore zeolite (a zeolite with a pore size of 0.44 to 0.65 nm formed mainly by a 10-membered ring) or large-pore zeolite (a zeolite with a pore size of 0.66 to 0.76 nm formed mainly by a 12-membered ring) where pores into which reactant molecules can diffuse three-dimensionally extend; in other words, where reactant molecules three-dimensionally diffuse in pores, for example, ZSM-5, MCM-22, β- and Y-type zeolites. As a zeolite catalyst component, a so-called high-silica zeolite may be preferable; specifically, a zeolite with a SiO₂/Al₂O₃ molar ratio of 10 to 150. When the zeolite catalyst component used is a high silica zeolite having active sites in a lower density in which diffusion of reactant molecules is limited, a polymerization reaction completes with a lower polymerization degree, giving a lower olefin comprising propylene or butene as a main component. The lower olefin thus produced can be easily released from a pore in the zeolite catalyst component, which is relatively larger and three-dimensionally extends, allowing a reactant molecule to diffuse. Then, the olefin is rapidly hydrogenated on the methanol synthesis catalyst component to become inactivated in further polymerization and thus to be stabilized. Use of the above-mentioned zeolites as a zeolite catalyst component makes it possible to produce propylene and/or butene and to produce propane and/or butane with a higher selectivity.

Furthermore, a catalyst for producing a liquefied petroleum gas according to the present invention has a longer catalyst life and is less deteriorated with time. When using the catalyst for producing a liquefied petroleum gas according to this invention, propane and/or butane, i.e., LPG can be produced with high activity and high yield for a longer period, in comparison with a catalyst comprising a Cu—Zn-based methanol synthesis catalyst and Y-type zeolite, for example. A catalyst for producing a liquefied petroleum gas comprising a Cu—Zn-based methanol synthesis catalyst as a methanol synthesis catalyst component is relatively unstable in a reaction atmosphere having higher concentrations of CO₂ and H₂O at high-temperature. Improvement in stability and extension of life of the catalyst are very important for practical application of a process for producing LPG from a synthesis gas and a process for producing LPG from a carbon-containing starting material such as a natural gas.

A nickel catalyst, for example, is widely used as a catalyst in a hydrogenation reaction of an olefin into a paraffin. But if a substance cannot act as a catalyst in a methanol synthesis reaction (CO+2H₂→CH₃OH), it is, of course, unpreferable as a methanol synthesis catalyst component used in this invention.

The reaction conditions are also important for stably producing LPG for a long period with a high conversion, a high selectivity and a high yield by reacting carbon monoxide and hydrogen in the presence of a catalyst for producing a liquefied petroleum gas of this invention. This invention may be particularly effective when carbon monoxide and hydrogen are reacted at a reaction temperature of 300° C. to 420° C. (both inclusive) and under a reaction pressure of 2.2 MPa to 10 MPa (both inclusive).

According to this invention, for example, the catalyst can exhibit such a high activity that a conversion of CO is 60% or more, specifically 70% or more, further specifically 80% or more, to give a hydrocarbon with the total content of propane and butane of 60% or more, specifically 70% or more, further specifically 75% or more.

Furthermore, according to this invention, LPG with the total content of propane and butane of 90 mol % or more, specifically 95 mol % or more (including 100 mol %), for example, can be produced. And, according to this invention, LPG with a content of propane of 50 mol % or more, specifically 60 mol % or more (including 100 mol %), for example, can be produced.

### BRIEF DESCRIPTION OF THE DRAWINGS

FIG. 1 is a process flow diagram showing a main configuration in an example of an LPG producing apparatus suitable for conducting the process for LPG production according to this invention.

### DESCRIPTION OF THE MAIN SYMBOLS

1: a reformer
1a: a reforming catalyst layer
2: a reactor
2a: a catalyst layer
3, 4, 5: lines.

### Best Mode for Carrying out the Invention

1. Catalyst for Producing a Liquefied Petroleum Gas According to the Present Invention

A catalyst for producing a liquefied petroleum gas according to the present invention comprises at least one methanol synthesis catalyst component in which an olefin-hydrogenation catalyst component is supported on a Zn—Cr-based methanol synthesis catalyst, and at least one zeolite catalyst component.

A catalyst for producing a liquefied petroleum gas of this invention can comprise other additive components as long as its intended effect would not be impaired.

A ratio of the methanol synthesis catalyst component to the zeolite catalyst component (methanol synthesis catalyst component/zeolite catalyst component; by weight) is preferably 0.1 or more, more preferably 0.5 or more. A ratio of the methanol synthesis catalyst component to the zeolite catalyst component (methanol synthesis catalyst component/zeolite catalyst component; by weight) is preferably 5 or less, more preferably 3 or less. By adjusting a ratio of the methanol synthesis catalyst component to the zeolite catalyst component within the above range, propane and/or butane can be produced with a higher selectivity and a higher yield.

A methanol synthesis catalyst component acts as a methanol synthesis catalyst and a hydrogenation catalyst for an olefin. A zeolite catalyst component acts as a solid acid zeolite catalyst, whose acidity is adjusted, in a condensation reaction of methanol into a hydrocarbon and/or a condensation reaction of dimethyl ether into a hydrocarbon. A ratio of the methanol synthesis catalyst component to the zeolite catalyst component is, therefore, reflected in a relative ratio of the ability to form methanol and the ability to hydrogenate an olefin to the ability to form a hydrocarbon from methanol, which the catalyst of this invention has. In this invention, when reacting carbon monoxide and hydrogen to produce a liquefied petroleum gas comprising propane or butane as a main component, carbon monoxide and hydrogen must be sufficiently converted into methanol by the action of a methanol synthesis catalyst component, and methanol produced must be sufficiently converted, by the action of a zeolite catalyst component, into an olefin comprising propylene or butene as a main component, which must be converted into a liquefied petroleum gas comprising propane or butane as a main component by the action of a methanol synthesis catalyst component.

By adjusting a ratio of the methanol synthesis catalyst component to the zeolite catalyst component (methanol synthesis catalyst component/zeolite catalyst component; by weight) to 0.1 or more, more preferably 0.5 or more, carbon monoxide and hydrogen can be converted into methanol with a higher conversion. Furthermore, by adjusting a ratio of the methanol synthesis catalyst component to the zeolite catalyst component (methanol synthesis catalyst component/zeolite catalyst component; by weight) to 0.8 or more, methanol produced can be converted into a liquefied petroleum gas comprising propane or butane as a main component with a higher selectivity.

On the other hand, by adjusting a ratio of the methanol synthesis catalyst component to the zeolite catalyst component (methanol synthesis catalyst component/zeolite catalyst component; by weight) to 5 or less, more preferably 3 or less, methanol produced can be converted into a liquefied petroleum gas comprising propane or butane as a main component with a higher conversion.

A ratio of the methanol synthesis catalyst component to the zeolite catalyst component is not limited to the above range, and can be appropriately determined, depending on the types of a methanol synthesis catalyst component and a zeolite catalyst component, and the like.

(Methanol Synthesis Catalyst Component)

A methanol synthesis catalyst component used in this invention is one in which an olefin-hydrogenation catalyst component is supported on a Zn—Cr-based methanol synthesis catalyst.

A Zn—Cr-based methanol synthesis catalyst component may be selected, without limitation, from those which contains Zn and Cr, and can act as a catalyst in the reaction: CO+2H₂ →CH₃OH, and a known Zn—Cr-based methanol synthesis catalyst or a commercially available Zn—Cr-based methanol synthesis catalyst can be used.

A Zn—Cr-based methanol synthesis catalyst is commonly a composite oxide containing Zn and Cr. The composite oxide may contain an element other than Zn, Cr and O, for example, Si, Al and so on.

A ratio of Zn to Cr in the Zn—Cr-based methanol synthesis catalyst (Zn/Cr; atomic ratio) is preferably 1 or more, more preferably 1.5 or more. A ratio of Zn to Cr in the Zn—Cr-based methanol synthesis catalyst (Zn/Cr; atomic ratio) is preferably 3 or less, more preferably 2.5 or less. By using a Zn—Cr-based methanol synthesis catalyst having a ratio of Zn to Cr within the above range, higher catalytic activity can be achieved, and propane and/or butane can be produced with a higher conversion, a higher selectivity and a higher yield.

Specific examples of a Zn—Cr-based methanol synthesis catalyst include KMA produced by Süd-Chemie Catalysts Japan, Inc.

The Zn—Cr-based methanol synthesis catalyst may be used alone or in combination of two or more.

An olefin-hydrogenation catalyst component may be selected, without limitation, from those which can act as a catalyst in a hydrogenation reaction of an olefin into a paraffin. Specific examples of an olefin-hydrogenation catalyst component include Fe, Co, Ni, Cu, Ru, Rh, Pd, Ir, Pt and so on. The olefin-hydrogenation catalyst components may be used alone or in combination of two or more.

Among them, a preferable olefin-hydrogenation catalyst component is Pd or Pt, more preferably Pd. By using Pd and/or Pt, more preferably Pd as an olefin-hydrogenation catalyst component, higher catalytic activity can be achieved, and propane and/or butane can be produced with a higher conversion, a higher selectivity and a higher yield.

Pd and Pt may not be necessarily contained as a metal, but can be contained in the form of an oxide, a nitrate, a chloride or the like. In such a case, it is preferred that the catalyst may be subjected to, for example, reduction by hydrogen before the reaction, to convert Pd and/or Pt into metallic palladium and/or metallic platinum, for achieving higher catalytic activity.

The reduction treatment condition for activating Pd and/or Pt can be determined, depending on some factors such as the types of a supported palladium compound and/or a supported platinum compound, as appropriate.

In the light of a more efficient hydrogenation of an olefin, an olefin-hydrogenation catalyst component such as Pd and Pt is preferably supported on a Zn—Cr-based methanol synthesis catalyst in a highly dispersed manner.

The total amount of the supported olefin-hydrogenation catalyst component in the methanol synthesis catalyst component is preferably 0.005 wt % or more, more preferably 0.01 wt % or more, particularly preferably 0.05 wt % or more, further preferably 0.1 wt % or more, further preferably 0.5 wt % or more. On the other hand, the total amount of the supported olefin-hydrogenation catalyst component in the methanol synthesis catalyst component is preferably 5 wt % or less, more preferably 3 wt % or less, in the light of dispersibility and economical efficiency. By adjusting the amount of a supported olefin-hydrogenation catalyst component in a methanol synthesis catalyst component within the above range, propane and/or butane can be produced with a higher conversion, a higher selectivity and a higher yield.

By adjusting the amount of a supported olefin-hydrogenation catalyst component to be 0.005 wt % or more, more preferably 0.5 wt % or more, carbon monoxide and hydrogen can be converted into methanol with a higher conversion, and methanol produced can be converted into a liquefied petroleum gas comprising propane or butane as a main component with a higher selectivity. On the other hand, by adjusting the amount of a supported olefin-hydrogenation catalyst component to be 5 wt % or less, methanol produced can be converted into a liquefied petroleum gas comprising propane or butane as a main component with a higher conversion. Furthermore, by adjusting the amount of a supported olefin-hydrogenation catalyst component to be 3 wt % or less, more preferably 2 wt % or less, the catalyst cost is reduced sufficiently.

A particularly preferable methanol synthesis catalyst component used in this invention is one in which Pd, preferably metallic Pd is supported on a Zn—Cr-based methanol synthesis catalyst.

In this methanol synthesis catalyst component, the amount of supported Pd is preferably 0.005 wt % or more, more preferably 0.01 wt % or more, particularly preferably 0.05 wt % or more, further preferably 0.1 wt % or more, further preferably 0.5 wt % or more. And, the amount of supported Pd is preferably 5 wt % or less, more preferably 4 wt % or less.

The above methanol synthesis catalyst component may be a Zn—Cr-based methanol synthesis catalyst on which other components, in addition to an olefin-hydrogenation catalyst component, are supported as long as the desired effects of the catalyst are maintained.

A methanol synthesis catalyst component in which an olefin-hydrogenation catalyst component such as Pd is supported on a Zn—Cr-based methanol synthesis catalyst can be prepared by a known method such as an impregnation method and a precipitation method. Sometimes, in comparison with a methanol synthesis catalyst component prepared by an impregnation method, a methanol synthesis catalyst component prepared by a precipitation method may exhibit a higher catalytic activity, and thus may allow an LPG production reaction to proceed at a lower reaction temperature, and a higher selectivity for a hydrocarbon and a higher selectivity for propane and butane may be achieved.

(Zeolite Catalyst Component)

A zeolite catalyst component may be selected, without limitation, from zeolites which can act as a catalyst in a condensation reaction of methanol into a hydrocarbon and/or a condensation reaction of dimethyl ether into a hydrocarbon, and a commercially available zeolite can be used.

The preferable zeolite catalyst components include a middle-pore or large-pore zeolite in which pores into which reactant molecules can diffuse extend three-dimensionally. Such zeolites include ZSM-5, MCM-22, β- and Y-type zeolites, for example. In this invention, a zeolite wherein reactant molecules three-dimensionally diffuse in pores including a middle-pore zeolite such as ZSM-5 and MCM-22 and a large-pore zeolite such as β- and Y-type zeolites, which generally have high selectivity in a condensation reaction of methanol and/or dimethyl ether into an alkyl-substituted aromatic hydrocarbon, are preferable to a small-pore zeolite such as SAPO-34 and a zeolite wherein reactant molecules do not three-dimensionally diffuse in pores such as mordenite, which generally have high selectivity in a condensation reaction of methanol and/or dimethyl ether into a lower olefin hydrocarbon. By using a zeolite wherein reactant molecules three-dimensionally diffuse in pores including a middle-pore zeolite and a large-pore zeolite, methanol formed may be converted into a olefin comprising propylene and/or butene as a main component, and further into a paraffin comprising propane and/or butane as a main component (a liquefied petroleum gas) with a higher selectivity.

Herein, a middle-pore zeolite means a zeolite with a pore size of 0.44 to 0.65 nm formed mainly by a 10-membered ring. And a large-pore zeolite means a zeolite with a pore size of 0.66 to 0.76 nm formed mainly by a 12-membered ring. A zeolite catalyst component more preferably has a pore size of 0.5 nm or more in view of selectivity for C3 component in the gaseous product. In addition, a zeolite catalyst component more preferably has a skeletal pore size of 0.76 nm or less in view of inhibiting the formation of the liquid product including an aromatic compound such as benzene and a gasoline component such as C5 component.

As a zeolite catalyst component, a so-called high-silica zeolite may be preferable; specifically, a zeolite with a SiO₂/Al₂O₃ molar ratio of 10 to 150 may be preferable. By using a high-silica zeolite with a SiO₂/Al₂O₃ molar ratio of 10 to 150 as a zeolite catalyst component, methanol produced may be converted into an olefin comprising propylene and/or butene as a main component, and further into a paraffin comprising propane and/or butane as a main component (a liquefied petroleum gas) with a higher selectivity. A SiO₂/Al₂O₃ molar ratio of a zeolite is more preferably 20 or more, particularly preferably 30 or more. And, a SiO₂/Al₂O₃ molar ratio of a zeolite is more preferably 100 or less, particularly preferably 50 or less.

A particularly preferable zeolite catalyst component is a middle-pore or large-pore zeolite with a SiO₂/Al₂O₃ molar ratio of 10 to 150 in which pores permitting diffusion of reactant molecules extend three-dimensionally. Such zeolites include a solid acid zeolite such as USY-type zeolite and high-silica-type β-zeolite, for example.

The above solid acid zeolite, whose acidity is adjusted by ion-exchange and the like, is used as a zeolite catalyst component.

A zeolite catalyst component may be selected from zeolites containing a metal such as alkali metals, alkaline earth metals and transition metals (for example, Pd); zeolites ion-exchanged with these metals or the like; and zeolites on which these metals or the like are supported. But a preferable zeolite catalyst component is a proton-type zeolite. By using a proton-type zeolite having a suitable acid strength and a suitable acidity (acid concentration), higher catalytic activity can be achieved, and propane and/or butane can be produced with a higher conversion and a higher selectivity.

A particularly preferable zeolite catalyst component is a proton-type β-zeolite with a SiO₂/Al₂O₃ molar ratio of 10 to 150, further preferably of 30 to 50.

Another preferable zeolite catalyst component is a Pd-supported β-zeolite with a SiO₂/Al₂O₃ molar ratio of 10 to 150, further preferably of 30 to 50, in which the amount of supported Pd is 3% by weight or less. The amount of supported Pd is preferably 1% by weight or less.

2. Process for Producing a Catalyst for Producing a Liquefied Petroleum Gas According to the Present Invention

A catalyst for producing a liquefied petroleum gas according to this invention is preferably produced by separately preparing a methanol synthesis catalyst component and a zeolite catalyst component, and then mixing them. By separately preparing a methanol synthesis catalyst component and a zeolite catalyst component, a composition, a structure and a property of each component can be easily optimized for each function. Generally, a methanol synthesis catalyst is required to be basic, while a zeolite catalyst is required to be acidic. Thus, optimization for each function may be difficult when both catalyst components are prepared all together.

A methanol synthesis catalyst component in which an olefin-hydrogenation catalyst component such as Pd is supported on a Zn—Cr-based methanol synthesis catalyst can be prepared by a known method such as an impregnation method and a precipitation method. A Zn—Cr-based methanol synthesis catalyst can be prepared by a known method, and a commercially available Zn—Cr-based methanol synthesis catalyst can be used.

Some of methanol synthesis catalyst components must be activated by reduction treatment before use, including those containing Pd as an oxide, nitrate or chloride. In this invention, it is not necessarily required to activate a methanol synthesis catalyst component by reduction treatment in advance. The methanol synthesis catalyst component can be activated by subjecting the catalyst for producing a liquefied petroleum gas of this invention to the reduction treatment, before the beginning of the reaction, after producing the catalyst by mixing a methanol synthesis catalyst component and a zeolite catalyst component, and then molding the mixture.

The reduction treatment condition can be determined, depending on some factors such as the type of the olefin-hydrogenation catalyst component in the methanol synthesis catalyst component, as appropriate.

A zeolite catalyst component can be prepared by a known method, and a commercially available zeolite can be used. A zeolite catalyst component can be, if necessary, subjected to acid property adjustment by, for example, metal-ion-exchange, before mixing with a methanol synthesis catalyst component.

A catalyst for producing a liquefied petroleum gas according to the present invention can be produced by homogeneously mixing a methanol synthesis catalyst component and a zeolite catalyst component, and then, if necessary, molding the mixture. A procedure of mixing and molding these catalyst components is not particularly limited, but is preferably a dry method. When mixing and molding these catalyst components by a wet method, there may occur a compound transfer between these catalyst components, for example, neutralization due to transfer of a basic component in a methanol synthesis catalyst component to an acidic site in a zeolite catalyst component, leading to the change of a property optimized for each function of these catalyst components, and the like. A catalyst can be molded by an appropriate method such as an extrusion and a tablet-compression.

In this invention, a methanol synthesis catalyst component and a zeolite catalyst component to be mixed preferably have a large particle size to some extent, and are preferably not powdery but granular.

Herein, “powder” means to have an average particle size of 10 μm or less, while “granule” means to have an average particle size of 100 μm or more.

A catalyst for producing a liquefied petroleum gas according to the present invention may be prepared by mixing a granular methanol synthesis catalyst component, which has an average particle size of 100 μm or more, and a granular zeolite catalyst component, which has an average particle size of 100 μm or more, and then, if necessary, molding the mixture, to give a catalyst having a further longer catalyst life with further less deterioration. Average particle sizes of a methanol synthesis catalyst component and a zeolite catalyst component to be mixed are more preferably 200 μm or more, particularly preferably 500 μm or more.

On the other hand, in the light of maintaining the excellent performance of the mixed catalyst of this invention, average particle sizes of a methanol synthesis catalyst component and a zeolite catalyst component to be mixed are preferably 5 mm or less, more preferably 2 mm or less.

It is preferable that a methanol synthesis catalyst component and a zeolite catalyst component to be mixed have the same average particle size.

In general, when preparing a mixed catalyst, each catalyst component is, if necessary, mechanically pulverized to the same average particle size of, for example, about 0.5 to 2 μm, then homogeneously mixed and then, if necessary, molded. Alternatively, all of catalyst components are put in a vessel, and mixed them, while mechanically pulverizing, until a homogeneous mixture with an average particle size of about 0.5 to 2 μm, for example, is obtained, and then, if necessary, the mixture is molded.

In contrast, when preparing a catalyst for producing a liquefied petroleum gas according to this invention by mixing a granular methanol synthesis catalyst component and a granular zeolite catalyst component, generally, each catalyst component is molded by a known molding method such as a tablet-compression and an extrusion, and, if necessary, mechanically pulverized to the same average particle size of preferably about 100 μm to 5 mm, and then these components are homogeneously mixed. Then, if necessary, the mixture is molded again to prepare a catalyst for producing a liquefied petroleum gas of this invention.

A catalyst for producing a liquefied petroleum gas according to the present invention may comprise other additive components, as necessary, as long as its intended effect would not be impaired.

3. Process for Producing a Liquefied Petroleum Gas

Next, there will be described a process for producing a liquefied petroleum gas comprising propane or butane, preferably propane, as a main component by reacting carbon monoxide and hydrogen using a catalyst for producing a liquefied petroleum gas according to this invention as described above.

A reaction temperature is preferably 300° C. or higher, more preferably 320° C. or higher, particularly preferably 340° C. or higher. By controlling a reaction temperature within the above range, propane and/or butane can be produced with a higher conversion and a higher yield.

On the other hand, a reaction temperature is preferably 420° C. or lower, more preferably 400° C. or lower, in the light of the restrictive temperature for the use of the catalyst and easy removal or recovery of the reaction heat.

A reaction pressure is preferably 2.2 MPa or higher, more preferably 2.5 MPa or higher, particularly preferably 3 MPa or higher. By controlling a reaction pressure within the above range, propane and/or butane can be produced with a higher conversion and a higher yield, and the deterioration with time of the catalyst can be reduced further, so that propane and/or butane can be produced for a further longer period with a higher conversion and a higher yield. In particular, by controlling a reaction pressure to be 3 MPa or higher, propane and/or butane can be produced with a sufficiently high conversion and a sufficiently high yield.

On the other hand, a reaction pressure is preferably 10 MPa or lower, more preferably 7 MPa or lower, in the light of economical efficiency.

A gas space velocity is preferably 500 hr⁻¹ or more, more preferably 1500 hr⁻¹ or more, in the light of economical efficiency. In addition, a gas space velocity is preferably 10000 hr⁻¹ or less, more preferably 5000 hr⁻¹ or less, in order that each of a methanol synthesis catalyst component and a zeolite catalyst component may have a contact time for achieving a further sufficient conversion.

A concentration of carbon monoxide in a gas fed into a reactor is preferably 20 mol % or more, more preferably 25 mol % or more, in the light of ensuring a pressure (partial pressure) of carbon monoxide required for the reaction, and improving a specific productivity of the materials. In addition, a concentration of carbon monoxide in a gas fed into a reactor is preferably 45 mol % or less, more preferably 40 mol % or less, in the light of a further sufficiently high conversion of carbon monoxide.

A concentration of hydrogen in a gas fed into a reactor is preferably 1.2 moles or more, more preferably 1.5 moles or more per one mole of carbon monoxide, in order that carbon monoxide may react more sufficiently. In addition, a concentration of hydrogen in a gas fed into a reactor is preferably 3 moles or less, more preferably 2.5 moles or less per one mole of carbon monoxide, in the light of economical efficiency. In some cases, a concentration of hydrogen in a gas fed into a reactor may be preferably reduced to about 0.5 moles per one mole of carbon monoxide.

A gas fed into a reactor may contain carbon dioxide in addition to carbon monoxide and hydrogen, which are starting materials of the reaction. By recycling carbon dioxide discharged from the reactor, or by adding the corresponding amount of carbon dioxide, formation of carbon dioxide from carbon monoxide by a shift reaction in the reactor can be substantially reduced or be eliminated.

A gas fed into a reactor can contain water vapor. And a gas fed into a reactor can contain an inert gas.

A gas fed into a reactor can be dividedly fed to the reactor so as to control a reaction temperature.

The reaction can be conducted in a fixed bed, a fluidized bed, a moving bed or the like, and can be preferably selected, taking both of control of a reaction temperature and a regeneration method of the catalyst into account. For example, a fixed bed may include a quench type reactor such as an internal multistage quench type, a multitubular type reactor, a multistage type reactor having a plurality of internal heat exchangers or the like, a multistage cooling radial flow type, a double pipe heat exchange type, an internal cooling coil type, a mixed flow type, and other types of reactors.

When used, a catalyst for producing a liquefied petroleum gas according to the present invention can be diluted with silica, alumina or an inert and stable heat conductor for controlling a temperature. In addition, when used, a catalyst for producing a liquefied petroleum gas according to the present invention can be applied to the surface of a heat exchanger for controlling a temperature.

4. Process for Producing a Liquefied Petroleum Gas from a Carbon-Containing Starting Material

In this invention, a synthesis gas can be used as a starting gas for producing a liquefied petroleum gas (LPG).

Next, there will be described an embodiment of a process for producing LPG according to this invention, comprising the steps of producing a synthesis gas from a carbon-containing starting material (synthesis gas production process) and then producing LPG from the obtained synthesis gas using a catalyst of this invention (liquefied petroleum gas production process).

<Synthesis Gas Production Process>

In a synthesis gas production process, a synthesis gas is produced from a carbon-containing starting material and at least one selected from the group consisting of H₂O, O₂ and CO₂.

A carbon-containing substance which can react with at least one selected from the group consisting of H₂O, O₂ and CO₂ to form H₂ and CO, can be used as a carbon-containing starting material. A substance known as a raw material for a synthesis gas can be used as a carbon-containing starting material; for example, lower hydrocarbons such as methane and ethane, a natural gas, a naphtha, a coal, and the like can be used.

Since a catalyst is generally used in a synthesis gas production process and a liquefied petroleum gas production process in this invention, a carbon-containing starting material (a natural gas, a naphtha, a coal and so on) preferably contains less catalyst poisoning components such as sulfur and a sulfur compound. When a carbon-containing starting material contains a catalyst poisoning component, a step of removing the catalyst poisoning component such as devulcanization can be conducted before a synthesis gas production process, if necessary.

A synthesis gas can be produced by reacting the above carbon-containing starting material with at least one selected from the group consisting of H₂O, O₂ and CO₂ in the presence of a catalyst for producing a synthesis gas (reforming catalyst).

A synthesis gas can be produced by a known method. When a natural gas (methane) is used as a starting material, for example, a synthesis gas can be produced by a water-vapor reforming method, an autothermal reforming method or the like. In these methods, water vapor required for a water-vapor reforming, oxygen required for an autothermal reforming, or the like can be fed, if necessary. When a coal is used as a starting material, a synthesis gas can be produced using an aerating gasification furnace, or the like.

For example, a shift reactor may be placed downstream of a reformer, which is a reactor for producing a synthesis gas from the above starting materials, so that a synthesis gas composition can be adjusted by a shift reaction (CO+H₂O→CO₂+H₂).

In this invention, a preferable composition of a synthesis gas produced in a synthesis gas production process is a molar ratio of H₂/CO is 7/3-2.3 in terms of the stoichiometry for a lower paraffin production, and a ratio of hydrogen to carbon monoxide (H₂/CO; by mole) in a synthesis gas produced is preferably 1.2 to 3. A ratio of hydrogen to carbon monoxide (H₂/CO; by mole) in a synthesis gas is preferably 1.2 or more, more preferably 1.5 or more, in order that carbon monoxide may react suitably, since hydrogen is generated by a shift reaction caused by water generated in a conversion reaction from a synthesis gas to LPG. It is only necessary to feed hydrogen in such an amount that carbon monoxide can react suitably to form a liquefied petroleum gas comprising propane or butane as a main component, and excessive hydrogen may increase the total pressure of a starting gas unnecessarily, leading to a lower economical efficiency. Thus, a ratio of hydrogen to carbon monoxide (H₂/CO; by mole) in a synthesis gas is preferably 3 or less, more preferably 2.5 or less.

A concentration of carbon monoxide in a synthesis gas produced is preferably 20 mol % or more, more preferably 25 mol % or more, in the light of ensuring a pressure (partial pressure) of carbon monoxide suitable for a conversion reaction from a synthesis gas to LPG, and improving a specific productivity of the materials. In addition, a concentration of carbon monoxide in a synthesis gas produced is preferably 45 mol % or less, more preferably 40 mol % or less, in the light of a further sufficiently high conversion of carbon monoxide in a conversion reaction from a synthesis gas to LPG.

A synthesis gas having the above composition can be produced by appropriately selecting reaction conditions such as a feeding ratio of a carbon-containing starting material and at least one material selected from the group consisting of steam (water), oxygen and carbon dioxide, a kind of a catalyst for producing a synthesis gas used, and the like.

For example, a synthesis gas can be produced using a gas whose composition is steam/methane (molar ratio) of 1 and carbon dioxide/methane (molar ratio) of 0.4 as a starting gas under the operation conditions of a reaction temperature (an outlet temperature of a catalyst layer) of 800 to 900° C., a reaction pressure of 1 to 4 MPa, a gas space velocity (GHSV) of 2000 hr⁻¹, in an external heating multitubular tubular-reactor type apparatus filled with a catalyst, a Ru or Rh/a sintered magnesia having the smaller surface area.

When using steam for reforming in a synthesis gas production, a ratio of steam/raw material carbon (S/C) is preferably 1.5 or less, more preferably 0.8 to 1.2, in the light of an energy efficiency. Meanwhile, such a low S/C value may lead to the considerable possibility of carbon precipitation formation.

When producing a synthesis gas with a low S/C, it may be preferable to use a catalyst which have a good activity of forming a synthesis gas and a low activity of forming a carbon precipitation, as described in, for example, WO 98/46524, Japanese Patent Laid-open Publication No. 2000-288394 and Japanese Patent Laid-open Publication No. 2000-469. Hereinafter, such a catalyst will be described.

The catalyst described in WO 98/46524 is a catalyst in which at least one catalyst metal selected from rhodium, ruthenium, iridium, palladium and platinum is supported on a support composed of a metal oxide, having a specific surface area of 25 m²/g or less, an electronegativity of a metal ion in the support metal oxide of 13.0 or less, and the amount of the supported catalyst metal of 0.0005 to 0.1 mol % to the support metal oxide in terms of metal. In the light of prevention of carbon precipitation, the electronegativity is preferably 4 to 12 and the specific surface area of the catalyst is preferably 0.01 to 10 m²/g.

An electronegativity of a metal ion in the metal oxide can be defined by the following equation:

Xi=(1+2i)Xo

wherein Xi represents an electronegativity of the metal ion; Xo represents an electronegativity of the metal; and i represents an electronic number of the metal ion.

When the metal oxide is a composite metal oxide, an average electronegativity of the metal ions is used, and the value is the sum total of electronegativity of the each metal ion in the composite metal oxide multiplied by a mole fraction of each oxide.

An electronegativity of a metal (Xo) is a Pauling's electronegativity. Pauling's electronegativities are listed in Table 15.4 in “Moore Physical Chemistry (latter volume) (4th edition), translated by Ryoichi Fujishiro, Tokyo Kagaku Dozin Co., Ltd., p. 707 (1974)”. An electronegativity of a metal ion (Xi) in a metal oxide is detailed in, for example, “Shokubai Koza (Lectures on Catalyst), Vol. 2, ed. the Catalysis Society of Japan, p. 145 (1985)”.

Examples of the metal oxide in this catalyst include those containing at least one metal such as Mg, Ca, Ba, Zn, Al, Zr and La. An example of such a metal oxide is magnesia (MgO).

In a process in which methane and steam are reacted (steam reforming), the reaction is represented by the following formula (I):

CH₄+H₂O [symbol] 3H₂+CO (i)

In a process in which methane and carbon dioxide are reacted (CO₂ reforming), the reaction is represented by the following formula (II):

CH₄+CO₂ [symbol] 2H₂+2CO (ii)

In a process in which methane, steam and carbon dioxide are reacted (steam/CO₂ mixed reforming), the reaction is represented by the following formula (iii):

3CH₄+2H₂O+CO₂ [symbol] 8H₂+4CO (iii)

For steam reforming using the above catalyst, a reaction temperature is preferably 600 to 1200° C., more preferably 600 to 1000° C., and a reaction pressure is preferably 0.098 MPaG to 3.9 MPaG, more preferably 0.49 MPaG to 2.9 MPaG (G indicates that the value is a gauge pressure). When the steam reforming is conducted with a fixed bed, a gas space velocity (GHSV) is preferably 1,000 to 10,000 hr⁻¹, more preferably 2,000 to 8,000 hr⁻¹. A rate of steam to a carbon-containing starting material is preferably 0.5 to 2 moles, more preferably 0.5 to 1.5 moles, further preferably 0.8 to 1.2 moles of steam (H₂O) per one mole of carbon in the carbon-containing starting material (excluding CO₂).

For CO₂ reforming using the above catalyst, a reaction temperature is preferably 500 to 1200° C., more preferably 600 to 1000° C., and a reaction pressure is preferably 0.49 MPaG to 3.9 MPaG, more preferably 0.49 MPaG to 2.9 MPaG. When the CO₂ reforming is conducted with a fixed bed, a gas space velocity (GHSV) is preferably 1,000 to 10,000 hr⁻¹, more preferably 2,000 to 8,000 hr⁻¹. A rate of CO₂ to a carbon-containing starting material is preferably 20 to 0.5 moles, more preferably 10 to 1 moles of CO₂ per one mole of carbon in the carbon-containing starting material (excluding CO₂).

When a carbon-containing starting material is reacted with a mixture of steam and CO₂ using the above catalyst to produce a synthesis gas (i.e., steam/CO₂ mixed reforming is conducted), there are no restrictions to a ratio of steam to CO₂, but generally a ratio of H₂O/CO₂ (molar ratio) is 0.1 to 10. A reaction temperature is preferably 550 to 1200° C., more preferably 600 to 1000° C., and a reaction pressure is preferably 0.29 MPaG to 3.9 MPaG, more preferably 0.49 MPaG to 2.9 MPaG. When the reaction is conducted with a fixed bed, a gas space velocity (GHSV) is preferably 1,000 to 10,000 hr⁻¹, more preferably 2,000 to 8,000 hr⁻¹. A rate of steam to a carbon-containing starting material is preferably 0.5 to 2 moles, more preferably 0.5 to 1.5 moles, further preferably 0.5 to 1.2 moles of steam (H₂O) per one mole of carbon in the carbon-containing starting material (excluding CO₂).

The catalyst described in Japanese Patent Laid-open Publication No. 2000-288394 is composed of a composite oxide having a composition represented by the following formula (I), characterized in that M¹ and Co are highly dispersed in the composite oxide:

a¹M¹·b¹Co c¹Mg·d¹Ca·e¹O (I)

wherein a¹, b¹, c¹, d¹ and e¹ are mole fractions, provided that a¹+b¹+c¹+d¹=1, 0.0001≦a¹≦0.10, 0.0001≦b¹≧0.20, 0.70≦(c¹+d¹)≦0.9998, 0<c¹<0.9998, 0≦d¹<0.9998, and e¹ is a number required for maintaining the charge balance of the elements and oxygen;

M¹ is at least one element selected from Group 6A elements, Group 7A elements, Group 8 transition elements except Co, Group 1B elements, Group 2B elements, Group 4B elements and lanthanoid elements in the Periodic Table.

The catalyst described in Japanese Patent Laid-open Publication No. 2000-469 is composed of a composite oxide having a composition represented by the following formula (II), characterized in that M² and Ni are highly dispersed in the composite oxide:

a²M²·b²Ni·c²Mg·d²Ca·e²O (II)

wherein a², b², c², d² and e² are mole fractions, provided that a²+b²+c²+d²=1, 0.0001≦a²≦0.10, 0.0001≦b2≦0.10, 0.80≦(c²+d²)≦0.9998, 0<c²≦0.9998, 0≦d²<0.9998, and e² is a number required for maintaining the charge balance of the elements and oxygen;

M² is at least one element selected from Group 3B elements, Group 4A elements, Group 6B elements, Group 7B elements, Group 1A elements and lanthanoid elements in the Periodic Table.

These catalysts can be used in the same way as the catalyst described in WO 98/46524.

A reforming reaction of a carbon-containing starting material, i.e., a reaction for producing a synthesis gas, is not limited to the above methods, and can be conducted in accordance with any of other known methods. A reforming reaction of a carbon-containing starting material can be conducted in various types of reactors, but is preferably conducted in a fixed bed or a fluidized bed.

<Liquefied Petroleum Gas Production Process>

In a liquefied petroleum gas production process, a lower-paraffin-containing gas, which comprises propane or butane as a main component of the hydrocarbon contained therein, is produced from the synthesis gas obtained in the above synthesis gas production process, using a catalyst for producing a liquefied petroleum gas according to the present invention. And then, water is separated from the lower-paraffin-containing gas produced, as necessary, and subsequently a low-boiling component having a lower boiling point or a lower sublimation point than the boiling point of propane (unreacted starting materials, hydrogen and carbon monoxide; by-products, carbon dioxide, ethane, ethylene and methane; and so on) and a high-boiling component having a higher boiling point than the boiling point of butane (by-products, high-boiling paraffin gases; and so on) are separated from the lower-paraffin-containing gas, as necessary, so as to obtain a liquefied petroleum gas (LPG) comprising propane or butane as a main component. If necessary, the gas may be pressurized and/or cooled so as to obtain a liquefied petroleum gas.

In a liquefied petroleum gas production process, carbon monoxide and hydrogen are reacted in the presence of the above catalyst for producing a liquefied petroleum gas of this invention, to produce a paraffin comprising propane or butane as a main component, preferably a paraffin comprising propane as a main component.

In this process, a gas fed into a reactor is the synthesis gas produced in the above synthesis gas production process. The gas fed into a reactor may contain, in addition to carbon monoxide and hydrogen, other components such as carbon dioxide, water, methane, ethane, ethylene and an inert gas. The gas fed into a reactor may be a gas obtained by adding carbon monoxide, hydrogen or other components, if necessary, to the synthesis gas produced in the above synthesis gas production process. And the gas fed into a reactor may be a gas obtained by separating a certain component, as necessary, from the synthesis gas produced in the above synthesis gas production process.

A gas fed into a reactor may comprise carbon dioxide, in addition to carbon monoxide and hydrogen, which are starting materials for producing a lower paraffin. As the carbon dioxide, by recycling carbon dioxide discharged from the reactor, or by using the corresponding amount of carbon dioxide, formation of carbon dioxide from carbon monoxide by a shift reaction in the reactor can be substantially reduced or be eliminated.

A gas fed into a reactor may comprise water vapor.

As described above, a reaction temperature is preferably 300° C. or higher, more preferably 320° C. or higher, particularly preferably 340° C. or higher. On the other hand, as described above, a reaction temperature is preferably 420° C. or lower, more preferably 400° C. or lower.

As described above, a reaction pressure is preferably 2.2 MPa or higher, more preferably 2.5 MPa or higher, particularly preferably 3 MPa or higher. On the other hand, as described above, a reaction pressure is preferably 10 MPa or lower, more preferably 7 MPa or lower.

As described above, a gas space velocity is preferably 500 hr⁻¹ or more, more preferably 1500 hr-1 or more. On the other hand, as described above, a gas space velocity is preferably 10000 hr⁻¹ or less, more preferably 5000 hr⁻¹ or less.

A gas fed into a reactor can be dividedly fed to the reactor so as to control a reaction temperature.

The reaction can be conducted in a fixed bed, a fluidized bed, a moving bed or the like, and can be preferably selected, taking both of control of a reaction temperature and a regeneration method of the catalyst into account. For example, a fixed bed may include a quench type reactor such as an internal multistage quench type, a multitubular type reactor, a multistage type reactor having a plurality of internal heat exchangers or the like, a multistage cooling radial flow type, a double pipe heat exchange type, an internal cooling coil type, a mixed flow type, and other types of reactors.

When used, a catalyst for producing a liquefied petroleum gas according to the present invention can be diluted with silica, alumina or an inert and stable heat conductor for controlling a temperature. In addition, when used, a catalyst for producing a liquefied petroleum gas according to the present invention can be applied to the surface of a heat exchanger for controlling a temperature.

A lower-paraffin-containing gas produced in the liquefied petroleum gas production process comprises a hydrocarbon containing propane or butane as a main component. In the light of liquefaction properties, it is preferable that the total content of propane and butane is higher in a lower-paraffin-containing gas. According to this invention, there can be obtained a lower-paraffin-containing gas having a content of propane and butane of 60% or more, preferably 70% or more, more preferably 75% or more (including 100%) on the basis of carbon to the hydrocarbon contained therein, in total.

Furthermore, a lower-paraffin-containing gas produced in the liquefied petroleum gas production process preferably contains more propane in comparison with butane, in the light of inflammability and vapor pressure properties.

A lower-paraffin-containing gas produced in a liquefied petroleum gas production process generally comprises water; a low-boiling component having a lower boiling point or a lower sublimation point than the boiling point of propane; and a high-boiling component having a higher boiling point than the boiling point of butane. Examples of a low-boiling component include ethane, methane and ethylene, which are by-products; carbon dioxide which is formed by a shift reaction; and hydrogen and carbon monoxide, which are unreacted starting materials. Examples of a high-boiling component include high-boiling paraffins (e.g., pentane, hexane and so on), which are by-products.

Thus, water, a low-boiling component and a high-boiling component are, as necessary, separated from a lower-paraffin-containing gas produced, so as to obtain a liquefied petroleum gas (LPG) comprising propane or butane as a main component.

Separation of water, a low-boiling component or a high-boiling component can be conducted in accordance with a known method.

Water can be separated by, for example, liquid-liquid separation.

A low-boiling component can be separated by, for example, gas-liquid separation, absorption separation or distillation; more specifically, gas-liquid separation at an ambient temperature under increased pressure, absorption separation at an ambient temperature under increased pressure, gas-liquid separation with cooling, absorption separation with cooling, or combination thereof. Alternatively, for this purpose, membrane separation or adsorption separation can be conducted, or these in combination with gas-liquid separation, absorption separation or distillation can be conducted. A gas recovery process commonly employed in an oil factory (described in “Oil Refining Processes”, ed. The Japan Petroleum Institute, Kodansha Scientific, 1998, pp. 28-32) can be applied to separation of a low-boiling component.

A preferable method of separation of a low-boiling component is an absorption process where a liquefied petroleum gas comprising propane or butane as a main component is absorbed into an absorbent liquid such as a high-boiling paraffin gas having a higher boiling point than butane, and a gasoline.

A high-boiling component can be separated by, for example, gas-liquid separation, absorption separation or distillation.

For consumer use, it is preferable that a content of a low-boiling component in the LPG is reduced to 5 mol % or less (including 0 mol %) by separation, for example, in the light of safety in use.

The total content of propane and butane in the LPG thus produced may be 90 mol % or more, more preferably 95 mol % or more (including 100 mol %). And a content of propane in the LPG produced may be 50 mol % or more, more preferably 60 mol % or more (including 100 mol %). Thus, according to this invention, LPG having a composition suitable for a propane gas, which is widely used as a fuel for household and business use, can be produced.

In this invention, a low-boiling component separated from the lower-paraffin-containing gas can be recycled as a starting material for the synthesis gas production process.

A low-boiling component separated from the lower-paraffin-containing gas comprises substances which can be recycled as starting materials for a synthesis gas production process; for example, methane, ethane, ethylene and so on. And carbon dioxide in the low-boiling component can be back to a synthesis gas by a CO₂ reforming reaction. In addition, a low-boiling component comprises hydrogen and carbon monoxide, which are unreacted starting materials. Therefore, the low-boiling component separated from the lower-paraffin-containing gas may be recycled as a starting material for a synthesis gas production process, leading to reduce a specific productivity of the materials.

The whole low-boiling components separated from a lower-paraffin-containing gas can be recycled to a synthesis gas production process. Alternatively, part of the low-boiling components may be removed outside the system, while the rest of low-boiling components may be recycled to a synthesis gas production process. A desired component can be separated from the low-boiling components and recycled to a synthesis gas production process.

In a synthesis gas production process, a content of a low-boiling component in a gas fed into a reformer, which is a reactor, in other words, a content of a recycled material may be determined as appropriate, and it may be, for example, 40 to 75 mol %.

For the purpose of recycling a low-boiling component, a known technique, e.g., providing a recycle line with a pressurization means can be appropriately employed.

<Process for Producing LPG>

Next, there will be described an embodiment of a process for producing LPG according to this invention with reference to the drawing.

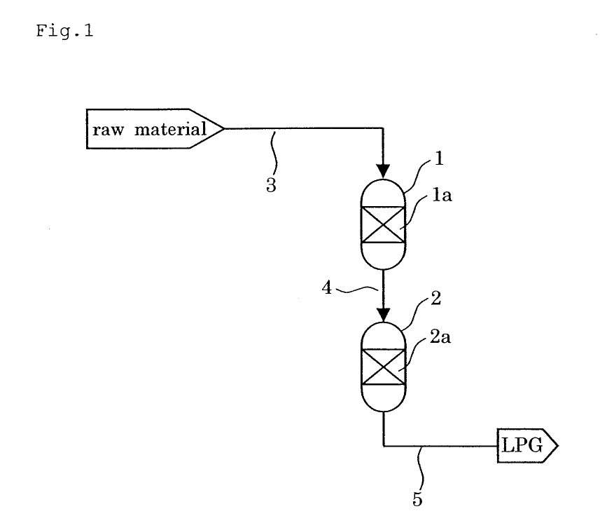

FIG. 1 shows an embodiment of an LPG production apparatus suitable for carrying out a production process for LPG according to this invention.

First, a natural gas (methane) as a carbon-containing starting material is fed into a reformer 1 via a line 3. And, for steam reforming, steam (not shown) is also fed into the line 3. In the reformer 1, there is a reforming catalyst layer 1a comprising a reforming catalyst (a catalyst for producing a synthesis gas). The reformer 1 also has a heating means for supplying heat required for reforming (not shown). In the reformer 1, methane is reformed in the presence of the reforming catalyst to produce a synthesis gas containing hydrogen and carbon monoxide.

The synthesis gas thus produced is fed into a reactor 2 via a line 4. In the reactor 2, there is a catalyst layer 2a comprising a catalyst of this invention. In the reactor 2, a hydrocarbon gas containing propane or butane as a main component (a lower-paraffin-containing gas) is produced from the synthesis gas in the presence of the catalyst of this invention.

The hydrocarbon gas thus produced is pressurized and cooled, after optional removal of water or the like, and LPG, which is a product, is obtained from a line 5. Optionally, hydrogen and the like may be removed from the LPG by, for example, gas-liquid separation.

The LPG production apparatus may be, as necessary, provided with a booster, a heat exchanger, a valve, an instrumentation controller and so on, which are not shown.

Alternatively, a gas obtained by adding carbon dioxide or the like to the synthesis gas produced in the reformer 1 may be fed into the reactor 2. And, a gas obtained by adding additional hydrogen or carbon monoxide to the synthesis gas produced in the reformer 1, or a gas obtained by adjusting its composition by a shift reaction, may be fed into the reactor 2.

Water, a low-boiling component, a high-boiling component, and the like may be separated from the hydrocarbon gas produced in the reactor 2, by a known method. The low-boiling component separated from the hydrocarbon gas may be recycled into the reformer 1 as a starting material for the synthesis gas production process (reforming process).

### EXAMPLES

The following will describe the present invention in more detail with reference to Examples. However, the present invention is not limited to these Examples.

### Example 1

### Preparation of a Catalyst

A mechanically powdered catalyst in which 1 wt % of Pd was supported on Zn—Cr-based methanol synthesis catalyst (also referred to as “Pd/Zn—Cr”; average particle size: 0.7 μm) was used as a methanol synthesis catalyst component. The catalyst was prepared as follows.

The Zn—Cr-based methanol synthesis catalyst used was KMA (trade name), produced by Süd-Chemie Catalysts Japan, Inc. (average particle size: about 1 mm). The composition of the Zn—Cr-based methanol synthesis catalyst is Zn/Cr=2 (atomic ratio).

First, to 4.4 mL of an aqueous solution of Pd(NH₃)₂(NO₃)₂ (Pd content: 4.558 wt %) was added 1 mL of ion-exchanged water, to obtain a Pd-containing solution. 20 g of the Zn—Cr-based methanol synthesis catalyst was added to the obtained Pd-containing solution, and impregnated with the Pd-containing solution. And then, the Zn—Cr-based methanol synthesis catalyst impregnated with the Pd-containing solution was dried in a drying oven at 120° C. for 12 hours, and calcined in an air at 450° C. for 2 hours. Subsequently, it was mechanically pulverized to give a methanol synthesis catalyst component.

A commercially available proton-type β-zeolite with a SiO₂/Al₂O₃ molar ratio of 37.1, produced by Tosoh Corporation, was mechanically pulverized to powder (average particle size: 0.7 μm) and used as a zeolite catalyst component.

The methanol synthesis catalyst component thus prepared and the zeolite catalyst component were homogeneously mixed with Pd/Zn—Cr: β-zeolite=2:1 (by weight). And, the mixture was molded by a tablet-compression and sized to give a granular molded catalyst having an average particle size of 1 mm.

(Production of LPG)

In a tubular reactor with an inner diameter of 6 mm was placed 1 g of the catalyst thus prepared, and the catalyst was reduced under a hydrogen stream at 400° C. for 3 hours before the beginning of the reaction.

After reduction treatment of the catalyst, a starting gas consisting of 66.7 mol % of hydrogen and 33.3 mol % of carbon monoxide (H₂/CO=2 (molar ratio)) was passed through the catalyst layer at a reaction temperature of 375° C., a reaction pressure of 5.1 MPa and a gas space velocity of 2000 hr⁻¹ (W/F=9.0 g·h/mol) to carry out the LPG production reaction. Gas chromatographic analysis of the product indicated that, after three hours from the beginning of the reaction, a conversion of carbon monoxide was 70.5%, a shift reaction conversion of carbon monoxide to carbon dioxide was 30.0%, and a conversion of carbon monoxide to a hydrocarbon was 40.5%. The produced hydrocarbon gas contained propane and butane in 75.0% on the basis of carbon, which consisted of 38.3% of propane and 61.7% of butane on the basis of carbon. And, after five hours from the beginning of the reaction, a conversion of carbon monoxide was 66.4%, a shift reaction conversion of carbon monoxide to carbon dioxide was 28.4%, and a conversion of carbon monoxide to a hydrocarbon was 38.0%. The produced hydrocarbon gas contained propane and butane in 74.8% on the basis of carbon, which consisted of 37.4% of propane and 62.6% of butane on the basis of carbon.

The results are shown in Table 1.

### Example 2

### Preparation of a Catalyst

A catalyst was prepared in the same way as Example 1, except that a methanol synthesis catalyst component and a zeolite catalyst component were separately molded by a tablet-compression to be a granule having an average particle size of 1 mm, without mechanically pulverizing, and then these components were mixed.

(Production of LPG)

Using the prepared catalyst, the LPG production reaction was carried out in the same way as Example 1. Gas chromatographic analysis of the product indicated that, after three hours from the beginning of the reaction, a conversion of carbon monoxide was 86.1%, a shift reaction conversion of carbon monoxide to carbon dioxide was 33.4%, and a conversion of carbon monoxide to a hydrocarbon was 52.7%. The produced hydrocarbon gas contained propane and butane in 81.8% on the basis of carbon, which consisted of 57.5% of propane and 42.5% of butane on the basis of carbon. And, after five hours from the beginning of the reaction, a conversion of carbon monoxide was 78.1%, a shift reaction conversion of carbon monoxide to carbon dioxide was 33.3%, and a conversion of carbon monoxide to a hydrocarbon was 44.8%. The produced hydrocarbon gas contained propane and butane in 77.2% on the basis of carbon, which consisted of 41.8% of propane and 58.2% of butane on the basis of carbon.

The results are shown in Table 1.

### Comparative Example 1

### Preparation of a Catalyst

A catalyst was prepared in the same way as Example 2, except that the Zn—Cr-based methanol synthesis catalyst (KMA (trade name), produced by Süd-Chemie Catalysts Japan, Inc.; also referred to as “Zn—Cr”) was used as a methanol synthesis catalyst component.

(Production of LPG)

Using the prepared catalyst, the LPG production reaction was carried out in the same way as Example 1. Gas chromatographic analysis of the product indicated that, after three hours from the beginning of the reaction, a conversion of carbon monoxide was 66.2%, a shift reaction conversion of carbon monoxide to carbon dioxide was 30.2%, and a conversion of carbon monoxide to a hydrocarbon was 36.0%. The produced hydrocarbon gas contained propane and butane in 75.4% on the basis of carbon, which consisted of 30.5% of propane and 69.5% of butane on the basis of carbon. And, after five hours from the beginning of the reaction, a conversion of carbon monoxide was 63.9%, a shift reaction conversion of carbon monoxide to carbon dioxide was 29.5%, and a conversion of carbon monoxide to a hydrocarbon was 34.3%. The produced hydrocarbon gas contained propane and butane in 71.6% on the basis of carbon, which consisted of 27.2% of propane and 72.8% of butane on the basis of carbon.

The results are shown in Table 1.

TABLE 1

| | Example 1 | | Example 2 | | Comp. Exam. 1 | |
|---|---|---|---|---|---|---|
| Catalyst | Pd/Zn—Cr | | Pd/Zn—Cr | | Zn—Cr | |
| | β-zeolite | | β-zeolite | | β-zeolite | |
| Average particle size of | 0.7 μm | | 1 mm | | 1 mm | |
| Catalyst components | | | | | | |
| Time on Stream (hr) | 3 | 5 | 3 | 5 | 3 | 5 |
| CO conversion (%) | 70.5 | 66.4 | 86.1 | 78.1 | 66.2 | 63.9 |
| CO₂ yield (%) | 30.0 | 28.4 | 33.4 | 33.3 | 30.2 | 29.5 |
| Hydrocarbon yield (%) | 40.5 | 38.0 | 52.7 | 44.8 | 36.0 | 34.3 |
| Product composition (%) | | | | | | |
| C1(methane) | 3.6 | 3.7 | 3.2 | 2.2 | 1.9 | 2.1 |
| C2(ethane) | 6.0 | 5.8 | 6.6 | 3.7 | 3.7 | 4.2 |
| C3(propane) | 28.7 | 28.0 | 47.1 | 32.3 | 23.0 | 19.5 |
| C4(butane) | 46.2 | 46.8 | 34.8 | 44.9 | 52.5 | 52.1 |
| C5(pentane) | 11.5 | 11.5 | 6.8 | 10.9 | 14.4 | 15.6 |
| C6(hexane) | 3.8 | 4.0 | 1.5 | 2.9 | 4.1 | 5.7 |
| C7(heptane) | 0.1 | 0.1 | 0.0 | 0.1 | 0.2 | 0.6 |
| C3 + C4 | 75.0 | 74.8 | 81.8 | 77.2 | 75.4 | 71.6 |
| C3/(C3 + C4) | 38.3 | 37.4 | 57.5 | 41.8 | 30.5 | 27.2 |

As seen in Table 1, Example 2 employing the catalyst of this invention consisting of Pd/Zn—Cr and β-zeolite exhibited a higher activity, a higher selectivity for a hydrocarbon, and a higher selectivity for propane and butane, in comparison with Comparative Example 1 employing the catalyst consisting of Zn—Cr and β-zeolite. Additionally, Example 1 employing the catalyst of this invention consisting of the powdery Pd/Zn—Cr and the powdery β-zeolite exhibited a higher activity, an equivalent or higher selectivity for a hydrocarbon, and an equivalent or higher selectivity for propane and butane, in comparison with Comparative Example 1 employing the catalyst consisting of the granular Zn—Cr and the granular O-zeolite.

Moreover, Example 2 employing the catalyst of this invention consisting of the granular Pd/Zn—Cr and the granular P-zeolite exhibited a higher activity, a higher selectivity for a hydrocarbon, and a higher selectivity for propane and butane, and the stability of the catalyst was higher, in comparison with Example 1 employing the catalyst of this invention consisting of the powdery Pd/Zn—Cr and the powdery O-zeolite.

### Comparative Example 2

### Preparation of a Catalyst

A commercially available Zn—Cr-based methanol synthesis catalyst (KMA (trade name), produced by Süd-Chemie Catalysts Japan, Inc.; also referred to as “Zn—Cr”) was used as a methanol synthesis catalyst component. The composition of the Zn—Cr-based methanol synthesis catalyst is Zn/Cr=2 (atomic ratio).

0.5 wt % of Pd was supported on a commercially available proton-type O-zeolite with a SiO₂/Al₂O₃ molar ratio of 37.1, produced by Tosoh Corporation, by an ion-exchange method, and the obtained 0.5 wt % Pd-supported O-zeolite (also referred to as “Pd-α-zeolite”) was used as a zeolite catalyst component.

And then, the methanol synthesis catalyst component and the zeolite catalyst component were homogeneously mixed with Zn—Cr:Pd-β-zeolite=2:1 (by weight). And, the mixture was molded by a tablet-compression and sized to give a granular molded catalyst having an average particle size of 1 mm.

(Production of LPG)

In a tubular reactor with an inner diameter of 6 mm was placed 1 g of the catalyst thus prepared, and the catalyst was reduced under a hydrogen stream at 400° C. for 3 hours before the beginning of the reaction.

After reduction treatment of the catalyst, a starting gas consisting of 66.7 mol % of hydrogen and 33.3 mol % of carbon monoxide (H₂/CO=2 (molar ratio)) was passed through the catalyst layer at a reaction temperature of 375° C., a reaction pressure of 2.1 MPa and a gas space velocity of 2000 hr⁻¹ (W/F=9.0 g·h/mol) to carry out the LPG production reaction. Gas chromatographic analysis of the product indicated that, after three hours from the beginning of the reaction, a conversion of carbon monoxide was 21.4%, a shift reaction conversion of carbon monoxide to carbon dioxide was 8.9%, and a conversion of carbon monoxide to a hydrocarbon was 12.5%. The produced hydrocarbon gas contained propane and butane in 76.3% on the basis of carbon, which consisted of 59.1% of propane and 40.9% of butane on the basis of carbon.

The results are shown in Table 2.

### Example 3

### Preparation of a Catalyst

A catalyst was prepared in the same way as Comparative Example 2, except that 0.5 wt % of Pd was supported on a Zn—Cr-based methanol synthesis catalyst (KMA (trade name), produced by Süd-Chemie Catalysts Japan, Inc.), and the obtained 0.5 wt % Pd-supported Zn—Cr-based methanol synthesis catalyst was used as a methanol synthesis catalyst component.

(Production of LPG)

Using the prepared catalyst, the LPG production reaction was carried out in the same way as Comparative Example 2. Gas chromatographic analysis of the product indicated that, after three hours from the beginning of the reaction, a conversion of carbon monoxide was 33.9%, a shift reaction conversion of carbon monoxide to carbon dioxide was 13.3%, and a conversion of carbon monoxide to a hydrocarbon was 20.6%. The produced hydrocarbon gas contained propane and butane in 80.2% on the basis of carbon, which consisted of 60.2% of propane and 39.8% of butane on the basis of carbon.

The results are shown in Table 2.

### Example 4

### Preparation of a Catalyst

A catalyst was prepared in the same way as Comparative Example 2, except that 1 wt % of Pd was supported on a Zn—Cr-based methanol synthesis catalyst (KMA (trade name), produced by Sud-Chemie Catalysts Japan, Inc.), and the obtained 1 wt % Pd-supported Zn—Cr-based methanol synthesis catalyst was used as a methanol synthesis catalyst component.

(Production of LPG)

Using the prepared catalyst, the LPG production reaction was carried out in the same way as Comparative Example 2. Gas chromatographic analysis of the product indicated that, after three hours from the beginning of the reaction, a conversion of carbon monoxide was 40.0%, a shift reaction conversion of carbon monoxide to carbon dioxide was 15.6%, and a conversion of carbon monoxide to a hydrocarbon was 24.4%. The produced hydrocarbon gas contained propane and butane in 79.3% on the basis of carbon, which consisted of 64.9% of propane and 35.1% of butane on the basis of carbon.

The results are shown in Table 2.

### Example 5

### Preparation of a Catalyst

A catalyst was prepared in the same way as Comparative Example 2, except that 2 wt % of Pd was supported on a Zn—Cr-based methanol synthesis catalyst (KMA (trade name), produced by Süsd-Chemie Catalysts Japan, Inc.), and the obtained 2 wt % Pd-supported Zn—Cr-based methanol synthesis catalyst was used as a methanol synthesis catalyst component.

(Production of LPG)

Using the prepared catalyst, the LPG production reaction was carried out in the same way as Comparative Example 2. Gas chromatographic analysis of the product indicated that, after three hours from the beginning of the reaction, a conversion of carbon monoxide was 44.4%, a shift reaction conversion of carbon monoxide to carbon dioxide was 18.6%, and a conversion of carbon monoxide to a hydrocarbon was 25.8%. The produced hydrocarbon gas contained propane and butane in 81.5% on the basis of carbon, which consisted of 61.4% of propane and 38.6% of butane on the basis of carbon.

The results are shown in Table 2.

### Example 6

### Preparation of a Catalyst

A catalyst was prepared in the same way as Comparative Example 2, except that 4 wt % of Pd was supported on a Zn—Cr-based methanol synthesis catalyst (KMA (trade name), produced by Süd-Chemie Catalysts Japan, Inc.), and the obtained 4 wt % Pd-supported Zn—Cr-based methanol synthesis catalyst was used as a methanol synthesis catalyst component.

(Production of LPG)

Using the prepared catalyst, the LPG production reaction was carried out in the same way as Comparative Example 2. Gas chromatographic analysis of the product indicated that, after three hours from the beginning of the reaction, a conversion of carbon monoxide was 45.7%, a shift reaction conversion of carbon monoxide to carbon dioxide was 19.3%, and a conversion of carbon monoxide to a hydrocarbon was 26.5%. The produced hydrocarbon gas contained propane and butane in 79.3% on the basis of carbon, which consisted of 62.7% of propane and 37.3% of butane on the basis of carbon.

The results are shown in Table 2.

TABLE 2

| | Comp. | Example | Example | Example | Example |
|---|---|---|---|---|---|
| | Exam. 2 | 3 | 4 | 5 | 6 |
| Amount of | 0 | 0.5 | 1 | 2 | 4 |
| Supported Pd | | | | | |
| in Pd/Zn—Cr | | | | | |
| (wt %) | | | | | |
| CO conversion | 21.4 | 33.9 | 40.0 | 44.4 | 45.7 |
| (%) | | | | | |
| CO₂ yield (%) | 8.9 | 13.3 | 15.6 | 18.6 | 19.3 |
| Hydrocarbon | 12.5 | 20.6 | 24.4 | 25.8 | 26.5 |
| yield (%) | | | | | |
| Product | | | | | |
| composition (%) | | | | | |
| C1(methane) | 5.0 | 3.8 | 5.5 | 4.2 | 5.6 |
| C2(ethane) | 11.5 | 8.9 | 9.4 | 7.9 | 8.6 |
| C3(propane) | 45.1 | 48.2 | 51.5 | 50.0 | 49.7 |
| C4(butane) | 31.2 | 31.9 | 27.8 | 31.4 | 29.6 |
| C5(pentane) | 5.4 | 5.4 | 4.4 | 5.0 | 4.9 |
| C6(hexane) | 1.8 | 1.6 | 1.4 | 1.5 | 1.6 |
| C7(heptane) | 0.0 | 0.0 | 0.0 | 0.0 | 0.0 |
| C3 + C4 | 76.3 | 80.2 | 79.3 | 81.5 | 79.3 |
| C3/(C3 + C4) | 59.1 | 60.2 | 64.9 | 61.4 | 62.7 |

As seen in Table 2, Examples 3 to 6 employing the catalyst of this invention consisting of Pd/Zn—Cr and Pd-β-zeolite exhibited a higher activity, an equivalent or higher selectivity for a hydrocarbon, and an equivalent or higher selectivity for propane and butane, in comparison with Comparative Example 2 employing the catalyst consisting of Zn—Cr and Pd-β-zeolite.

### Example 7

### Preparation of a Catalyst

1 wt % of Pd was supported on the Zn—Cr-based methanol synthesis catalyst (KMA (trade name), produced by Süd-Chemie Catalysts Japan, Inc.) by an impregnation method, and the obtained 1 wt % Pd-supported Zn—Cr-based methanol synthesis catalyst was used as a methanol synthesis catalyst component. The catalyst was prepared as follows.

First, 1.1 mL of an aqueous solution of Pd(NO₃)₂ (Pd(NO₃)₂ content: 10 wt %) was prepared. 5 g of the Zn—Cr-based methanol synthesis catalyst was added to the obtained Pd-containing solution, and impregnated with the Pd-containing solution. And then, the Zn—Cr-based methanol synthesis catalyst impregnated with the Pd-containing solution was dried in a drying oven at 120° C. for 12 hours, and calcined in an air at 300° C. for 4 hours. Subsequently, it was mechanically pulverized to give a methanol synthesis catalyst component.

0.5 wt % of Pd was supported on a commercially available proton-type β-zeolite with a SiO₂/Al₂O₃ molar ratio of 37.1, produced by Tosoh Corporation, by an ion-exchange method, and the obtained 0.5 wt % Pd-supported P-zeolite was used as a zeolite catalyst component.

The methanol synthesis catalyst component and the zeolite catalyst component were homogeneously mixed with Pd/Zn—Cr:Pd-β-zeolite=2:1 (by weight). And, the mixture was molded by a tablet-compression and sized to give a granular molded catalyst having an average particle size of 1 mm.

(Production of LPG)

In a tubular reactor with an inner diameter of 6 mm was placed 1 g of the catalyst thus prepared, and the catalyst was reduced under a hydrogen stream at 400° C. for 3 hours before the beginning of the reaction.

After reduction treatment of the catalyst, a starting gas consisting of 66.7 mol % of hydrogen and 33.3 mol % of carbon monoxide (H₂/CO=2 (molar ratio)) was passed through the catalyst layer at a reaction temperature of 375° C., a reaction pressure of 2.1 MPa and a gas space velocity of 2000 hr⁻¹ (W/F=9.0 g·h/mol) to carry out the LPG production reaction. Gas chromatographic analysis of the product indicated that, after three hours from the beginning of the reaction, a conversion of carbon monoxide was 40.8%, a shift reaction conversion of carbon monoxide to carbon dioxide was 16.2%, and a conversion of carbon monoxide to a hydrocarbon was 24.6%. The produced hydrocarbon gas contained propane and butane in 77.4% on the basis of carbon.

The results are shown in Table 3.

### Example 8

### Preparation of a Catalyst

A catalyst was prepared in the same way as Example 7, except that in the preparation of the methanol synthesis catalyst component, 1 wt % of Pd was supported on the Zn—Cr-based methanol synthesis catalyst (KMA (trade name), produced by Süd-Chemie Catalysts Japan, Inc.) by a precipitation method as follows.

First, in a beaker were placed 1.1 mL of an aqueous solution of Pd(NO₃)₂ (Pd(NO₃)₂ content: 10 wt %) and 150 mL of water, and the resulting solution was stirred. While stirring the solution, the Zn—Cr-based methanol synthesis catalyst (particle size: 105 μm or less) was added thereto. And then, a 0.25 M NaCO₃ aqueous solution was added drop by drop to the solution containing Zn—Cr powder so that pH of the solution is 10. Subsequently, the resulting material was filtrated and washed with ion-exchanged water. And then, the resulting Pd-supported Zn—Cr-based methanol synthesis catalyst was dried at 120° C. for 12 hours, and calcined in an air at 300° C. for 4 hours.

(Production of LPG)

Using the prepared catalyst, the LPG production reaction was carried out in the same way as Example 7. Gas chromatographic analysis of the product indicated that, after three hours from the beginning of the reaction, a conversion of carbon monoxide was 44.0%, a shift reaction conversion of carbon monoxide to carbon dioxide was 17.6%, and a conversion of carbon monoxide to a hydrocarbon was 26.4%. The produced hydrocarbon gas contained propane and butane in 78.9% on the basis of carbon.

The results are shown in Table 3.

TABLE 3

| | Example 7 | Example 8 |
|---|---|---|
| Method of supporting Pd | Impregnation | Precipitation |
| for Pd/Zn—Cr | method | method |
| CO conversion (%) | 40.8 | 44.0 |
| CO₂ yield (%) | 16.2 | 17.6 |
| Hydrocarbon yield (%) | 24.6 | 26.4 |
| Product composition (%) | | |
| C1(methane) | 8.3 | 7.2 |
| C2(ethane) | 11.7 | 9.2 |
| C3(propane) | 59.6 | 50.0 |
| C4(butane) | 17.8 | 28.7 |
| C5(pentane) | 2.1 | 4.1 |
| C6(hexane) | 0.6 | 1.1 |
| C7(heptane) | 0.6 | 0.0 |
| C3 + C4 | 77.4 | 78.9 |

As seen in Table 3, Examples 8 in which the methanol synthesis catalyst component, Pd/Zn—Cr, was prepared by a precipitation method exhibited a higher activity, in comparison with Example 7 in which Pd/Zn—Cr was prepared by an impregnation method.

### INDUSTRIAL APPLICABILITY

As described above, a catalyst for producing a liquefied petroleum gas according to the present invention is a less deteriorative catalyst with a longer catalyst life, which enables the production of a hydrocarbon containing propane or butane as a main component, i.e., a liquefied petroleum gas (LPG), by reacting carbon monoxide and hydrogen, with high activity, high selectivity and high yield. Therefore, by using the catalyst of this invention, propane and/or butane can be stably produced for a long period with high activity, high selectivity and high yield, from a carbon-containing starting material such as a natural gas or a synthesis gas. In other words, by using the catalyst of this invention, a liquefied petroleum gas with a high concentration of propane and/or butane can be stably produced for a long period with high yield, from a carbon-containing starting material such as a natural gas or a synthesis gas.

1. A catalyst for producing a liquefied petroleum gas, which is used for producing a liquefied petroleum gas containing propane or butane as a main component by reacting carbon monoxide and hydrogen, comprising
a methanol synthesis catalyst component in which an olefin-hydrogenation catalyst component is supported on a Zn—Cr-based methanol synthesis catalyst; and
a zeolite catalyst component.

2. The catalyst according to claim 1, wherein a ratio (by weight) of the methanol synthesis catalyst component to the zeolite catalyst component [(methanol synthesis catalyst component)/(zeolite catalyst component)] is 0.1 to 5.

3. The catalyst according to claim 1, wherein the total amount of the supported olefin-hydrogenation catalyst component in the methanol synthesis catalyst component is 0.005 to 5% by weight.

4. The catalyst according to claim 1, wherein the Zn—Cr-based methanol synthesis catalyst is a composite oxide containing Zn and Cr.

5. The catalyst according to claim 4, wherein a ratio of Zn to Cr (Zn/Cr) in the Zn—Cr-based methanol synthesis catalyst is 1 to 3 (atomic ratio).

6. The catalyst according to claim 1, wherein the olefin-hydrogenation catalyst component is at least one selected from the group consisting of Fe, Co, Ni, Cu, Ru, Rh, Pd, Ir and Pt.

7. The catalyst according to claim 6, wherein the olefin-hydrogenation catalyst component is Pd.

8. The catalyst according to claim 7, wherein the amount of supported Pd in the methanol synthesis catalyst component is 0.005 to 5% by weight.

9. The catalyst according claim 1, wherein the zeolite catalyst component is a β-zeolite.

10. The catalyst according to claim 9, wherein the P-zeolite is a proton-type β-zeolite with a SiO₂/Al₂O₃ molar ratio of 10 to 150.

11. The catalyst according to claim 9, wherein the β-zeolite is a Pd-supported β-zeolite with a SiO₂/Al₂O₃ molar ratio of 10 to 150 in which the amount of supported Pd is 3% by weight or less.

12. A process for producing a liquefied petroleum gas, comprising a step of:
reacting carbon monoxide and hydrogen in the presence of the catalyst according to claim 1, whereby producing a liquefied petroleum gas containing propane or butane as a main component.

13. The process for producing a liquefied petroleum gas according to claim 12, wherein a reaction temperature in the reaction of carbon monoxide and hydrogen is in a range of 300° C. to 420° C.

14. The process for producing a liquefied petroleum gas according to claim 12, wherein a reaction pressure in the reaction of carbon monoxide and hydrogen is in a range of 2.2 MPa to 10 MPa.

15. A process for producing a liquefied petroleum gas, comprising a step of:
feeding a synthesis gas to a catalyst layer comprising the catalyst according to claim 1, whereby producing a liquefied petroleum gas containing propane or butane as a main component.

16. A process for producing a liquefied petroleum gas, comprising:
(1) a step of producing a synthesis gas from a carbon-containing starting material and at least one selected from the group consisting of H₂O, O₂ and CO₂; and
(2) a step of producing a liquefied petroleum gas wherein the synthesis gas is fed to a catalyst layer comprising the according to claim 1, whereby producing a liquefied petroleum gas containing propane or butane as a main component.
